(19) United States
(12) Patent Application Publication
(10) Pub. No.: US 2026/0248566 A1
(43) Pub. Date: Aug. 27, 2026

(54) AUTOMATED DETERMINATION OF DEPLOYMENT SETTINGS FOR A COMPUTER-ASSISTED SYSTEM

(71) Applicant: INTUITIVE SURGICAL OPERATIONS, INC., Sunnyvale, CA (US)

(72) Inventors: Russell PARRISH, Campbell, CA (US); Pratap GANAPATHY, Menlo Park, CA (US); Mark GARIBALDI, Montara, CA (US); Craig GOTSILL, San Francisco, CA (US); Helene HAUGERUD, San Francisco, CA (US); Ross LOCKWOOD, San Francisco, CA (US); Dinesh RABINDRAN, San Jose, CA (US); Kelli SUM, San Jose, CA (US); Prasad V. UPADRASTA, San Jose, CA (US)

(21) Appl. No.: 19/163,546

(22) PCT Filed: Apr. 5, 2024
(86) PCT No.: PCT/US2024/023220

§ 371 Date: Sep. 9, 2025

Related U.S. Application Data
(60) Provisional application No. 63/494,919, filed on Apr. 7, 2023.

(51) Int. Cl. A61B 34/30; A61B 34/20; A61B 90/00

(52) U.S. Cl. CPC A61B 34/30; A61B 34/20; A61B 90/361; A61B 2034/2065

(57) ABSTRACT

A computer-assisted device includes a repositionable structure and a processing system. The processing system is configured to perform the steps of determining a location of the repositionable structure relative to a patient on an operating support; determining an imaging device orientation of an imaging device relative to the patient; determining a deployment for the repositionable structure based on the imaging device orientation and the location of the repositionable structure relative to the patient; and causing one or more instructions for configuring the repositionable structure to be displayed.

### RELATED APPLICATIONS

This application claims the benefit of U.S. Provisional Application No. 63/494,919, filed Apr. 7, 2023, and entitled “Automated Determination of Deployment Settings for a Computer-assisted System,” which is incorporated by reference herein.

### TECHNICAL FIELD

The present disclosure relates generally to operation of computer-assisted systems with repositionable structures, such as articulated arms, and more particularly to automated determination of deployment settings for such a computer-assisted system.

### BACKGROUND

Computer-assisted electronic systems are being used more and more often. This is especially true in industrial, entertainment, educational, and other settings. As a medical example, the medical facilities of today have large arrays of electronic systems being found in operating rooms, interventional suites, intensive care wards, emergency rooms, and/or the like. Many of these electronic systems may be capable of autonomous or semi-autonomous motion. It is also known for personnel to control the motion and/or operation of electronic systems using one or more input devices located at a user control system. As a specific example, minimally invasive, robotic telesurgical systems permit surgeons to operate on patients from bedside or remote locations. Telesurgery refers generally to surgery performed using surgical systems where the surgeon uses some form of remote control, such as a servomechanism, to manipulate surgical instrument movements rather than directly holding and moving the instruments by hand.

When a computer-assisted system is used to perform a task at a worksite (e.g., an interior anatomy of a patient in a medical example), the computer-assisted system needs to be positioned within a sterile environment and oriented toward the patient so that the repositionable structures of the computer-assisted system can have suitable range of motion to control one or more instruments to access the worksite. Ideally, the computer-assisted system is positioned so that some or all joints of each repositionable structure are located at or near a center of their respective ranges of motion when configured to control the one or more instruments when the instruments are used to access a target anatomy of the patient, who is located within the worksite located on the sterile side of the computer-assisted system. Generally, the surgeon who will operate the computer-assisted system can best determine what target anatomy within the worksite is most suitable for a particular procedure.

Accordingly, improved techniques for implementing deployment of a computer-assisted system are desirable.

### SUMMARY

In some embodiments, a computer-assisted system includes a repositionable structure and a processing system. In the embodiments, the processing system is configured to perform the steps of determining a location of the repositionable structure relative to a patient on an operating support; determining an imaging device orientation of an imaging device relative to the patient; determining a deployment for the repositionable structure based on the imaging device orientation and the location of the repositionable structure relative to the patient; and causing one or more instructions for configuring the repositionable structure to be displayed.

In some embodiments, a method includes determining a location of a repositionable structure of a computer-assisted system relative to a patient on an operating support; determining an imaging device orientation of an imaging device relative to the patient; determining a deployment for the repositionable structure based on the imaging device orientation and the location of the repositionable structure relative to the patient; and causing one or more instructions for configuring the repositionable structure to be displayed.

In some embodiments a non-transitory machine-readable medium includes a plurality of machine-readable instructions which when executed by one or more processors associated with a computer-assisted device are adapted to cause the one or more processors to perform the methods disclosed herein.

The foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

FIG. 1 is a simplified diagram of an example a computer-assisted system according to some embodiments.

FIG. 2 is a simplified diagram of a follower device with a sensor system according to some embodiments.

FIG. 3 is a simplified diagram of a follower device with a display system according to some embodiments.

FIG. 4 is a simplified diagram of a method for deploying a computer-assisted system according to some embodiments.

FIG. 5 is a simplified diagram of a patient positioned on an operating support according to some embodiments.

FIG. 6 is a simplified diagram of a target anatomy and an associated workspace according to some embodiments.

FIG. 7 is a simplified diagram of various locations proximate an operating support at which a follower device of a computer-assisted system can be positioned according to some embodiments.

FIG. 8 is a simplified diagram of a manually controlled imaging device inserted into a worksite according to some embodiments.

FIG. 9 is a simplified diagram of a manually controlled imaging device being directed toward a target anatomy according to some embodiments.

FIGS. 10A-10D are simplified diagrams of various deployments selected by the method of FIG. 4 according to some embodiments.

FIG. 11 is a simplified diagram of a method for selecting a deployment for a computer-assisted system according to some embodiments.

FIG. 12 is a simplified diagram of a method for selecting a deployment for a computer-assisted system according to some embodiments.

In the figures, elements having the same designations have the same or similar functions.

### DETAILED DESCRIPTION

This description and the accompanying drawings that illustrate inventive aspects, embodiments, embodiments, or modules should not be taken as limiting-the claims define the protected invention. Various mechanical, compositional, structural, electrical, and operational changes may be made without departing from the spirit and scope of this description and the claims. In some instances, well-known circuits, structures, or techniques have not been shown or described in detail in order not to obscure the invention. Like numbers in two or more figures represent the same or similar elements.

In this description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional.

Further, the terminology in this description is not intended to limit the invention. For example, spatially relative terms-such as “beneath”, “below”, “lower”, “above”, “upper”, “proximal”, “distal”, and the like-may be used to describe one element's or feature's relationship to another element or feature as illustrated in the figures. These spatially relative terms are intended to encompass different positions (i.e., locations) and orientations (i.e., rotational placements) of the elements or their operation in addition to the position and orientation shown in the figures. For example, if the content of one of the figures is turned over, elements described as “below” or “beneath” other elements or features would then be “above” or “over” the other elements or features. Thus, the exemplary term “below” can encompass both positions and orientations of above and below. A device may be otherwise oriented (rotated 90 degrees or at other orientations) and the spatially relative descriptors used herein interpreted accordingly. Likewise, descriptions of movement along and around various axes include various special element positions and orientations. In addition, the singular forms “a”, “an”, and “the” are intended to include the plural forms as well, unless the context indicates otherwise. And, the terms “comprises”, “comprising”, “includes”, and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components.

Elements described in detail with reference to one embodiment, embodiment, or module may, whenever practical, be included in other embodiments, embodiments, or modules in which they are not specifically shown or described. For example, if an element is described in detail with reference to one embodiment and is not described with reference to a second embodiment, the element may nevertheless be claimed as included in the second embodiment. Thus, to avoid unnecessary repetition in the following description, one or more elements shown and described in association with one embodiment, embodiment, or application may be incorporated into other embodiments, embodiments, or aspects unless specifically described otherwise, unless the one or more elements would make an embodiment or embodiment non-functional, or unless two or more of the elements provide conflicting functions.

In some instances, well known methods, procedures, components, and circuits have not been described in detail so as not to unnecessarily obscure aspects of the embodiments.

This disclosure describes various elements (such as systems and devices, and portions of systems and devices) with examples in three-dimensional space. In such examples, the term “position” refers to the location of an element or a portion of an element in a three-dimensional space (e.g., three degrees of translational freedom along Cartesian x-, y-, and z-coordinates). Also in such examples, the term “orientation” refers to the rotational placement of an element or a portion of an element (three degrees of rotational freedom-e.g., roll, pitch, and yaw). Other examples may encompass other dimensional spaces, such as two-dimensional spaces. As used herein, the term “pose” refers to the position, the orientation, or the position and the orientation combined, of an element or a portion of an element. As used herein, and for an element or portion of an element of a structure or assembly (e.g., of a computer-assisted system or a repositionable structure, etc.), the term “proximal” in a kinematic series refers to a direction toward the base of the kinematic series, and the term “distal” refers to a direction away from the base along the kinematic series.

Aspects of this disclosure are described in reference to electronic systems, computer-assisted devices, and robotic devices, which may include systems and devices that are teleoperated, remote-controlled, autonomous, semiautonomous, manually manipulated, and/or the like. Example computer-assisted systems include those that comprise robots or robotic devices. Further, aspects of this disclosure are described in terms of an embodiment using a medical system, such as the da Vinci® Surgical System commercialized by Intuitive Surgical, Inc. of Sunnyvale, California. Knowledgeable persons will understand, however, that inventive aspects disclosed herein may be embodied and implemented in various ways, including robotic and, if applicable, non-robotic embodiments. Embodiments described for da Vinci® Surgical Systems are merely exemplary, and are not to be considered as limiting the scope of the inventive aspects disclosed herein. For example, techniques described with reference to surgical instruments and surgical methods may be used in other contexts. Thus, the instruments, systems, and methods described herein may be used for humans, animals, portions of human or animal anatomy, industrial systems, general robotic, or teleoperational systems. As further examples, the instruments, systems, and methods described herein may be used for non-medical purposes including industrial uses, general robotic uses, sensing or manipulating non-tissue work pieces, cosmetic improvements, imaging of human or animal anatomy, gathering data from human or animal anatomy, setting up or taking down systems, training medical or non-medical personnel, and/or the like. Additional example applications include use for procedures on tissue removed from human or animal anatomies (with or without return to a human or animal anatomy) and for procedures on human or animal cadavers. Further, these techniques can also be used for medical treatment or diagnosis procedures that include, or do not include, surgical aspects.

### Example Computer-Assisted System

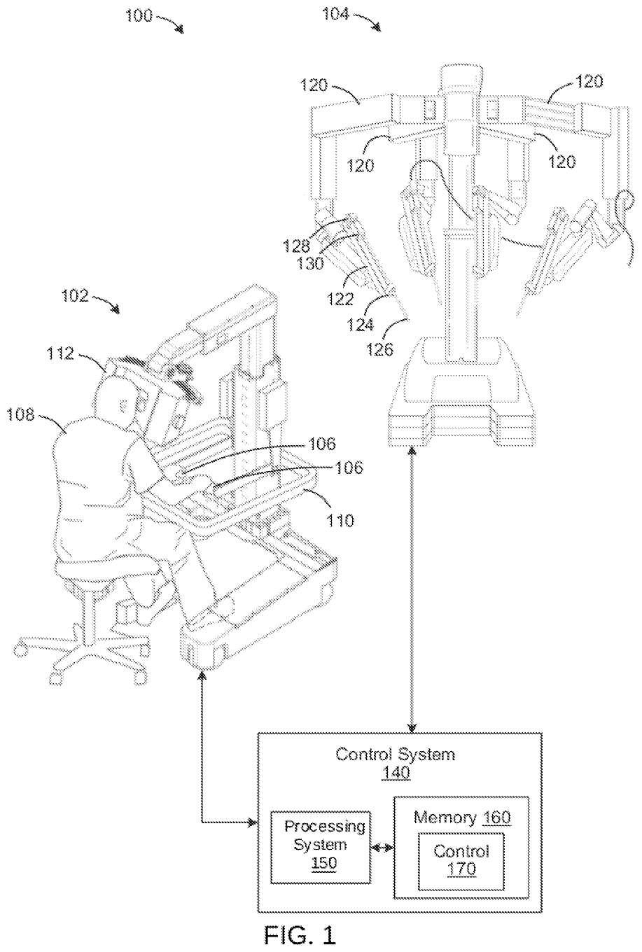

FIG. 1 is a simplified diagram of an example computer-assisted system 100 according to some embodiments. In some examples, the computer-assisted system 100 is a teleoperated system. In medical examples, computer-assisted system 100 can be a teleoperated medical system such as a surgical system. As shown, computer-assisted system 100 includes a follower device 104 that can be teleoperated by being controlled by one or more leader devices (also called “leader input devices” when designed to accept external input), described in greater detail below. Systems that include a leader device and a follower device are referred to as leader-follower systems, and also sometimes referred to as master-slave systems. Also shown in FIG. 1 is an input system that includes a workstation 102 (e.g., a console), and in various embodiments the input system can be in any appropriate form and may or may not include a workstation 102.

In the example of FIG. 1, workstation 102 includes one or more leader input devices 106 that are designed to be contacted and manipulated by an operator 108. For example, workstation 102 can comprise one or more leader input devices 106 for use by the hands, the head, or some other body part(s) of operator 108. Leader input devices 106 in this example are supported by workstation 102 and can be mechanically grounded. In some embodiments, an ergonomic support 110 (e.g., forearm rest) can be provided on which operator 108 can rest his or her forearms. In some examples, operator 108 can perform tasks at a worksite near follower device 104 during a procedure by commanding follower device 104 using leader input devices 106.

A display unit 112 is also included in workstation 102. Display unit 112 can display images for viewing by operator 108. Display unit 112 can be moved in various degrees of freedom to accommodate the viewing position of operator 108 and/or to optionally provide control functions as another leader input device. In the example of computer-assisted system 100, displayed images can depict a worksite at which operator 108 is performing various tasks by manipulating leader input devices 106 and/or display unit 112. In some examples, images displayed by display unit 112 can be received by workstation 102 from one or more imaging devices arranged at a worksite. In other examples, the images displayed by display unit 112 can be generated by display unit 112 (or by a different connected device or system), such as for virtual representations of tools, the worksite, or for user interface components.

When using workstation 102, operator 108 can sit in a chair or other support in front of workstation 102, position his or her eyes in front of display unit 112, manipulate leader input devices 106, and rest his or her forearms on ergonomic support 110 as desired. In some embodiments, operator 108 can stand at the workstation or assume other poses, and display unit 112 and leader input devices 106 can be adjusted in position (height, depth, etc.) to accommodate operator 108.

In some embodiments, the one or more leader input devices 106 can be ungrounded (ungrounded leader input devices being not kinematically grounded, such as leader input devices held by the hands of operator 108 without additional physical support). Such ungrounded leader input devices can be used in conjunction with display unit 112. In some embodiments, operator 108 can use a display unit 112 positioned near the worksite, such that operator 108 manually operates instruments at the worksite, such as a laparoscopic instrument in a surgical example, while viewing images displayed by display unit 112.

Computer-assisted system 100 can also include follower device 104, which can be commanded by workstation 102. In a medical example, follower device 104 can be located near an operating table (e.g., a table, bed, or other support) on which a patient can be positioned. In some medical examples, the worksite is provided on an operating table, e.g., on or in a patient, simulated patient, or model, etc. (not shown). The follower device 104 shown includes a plurality of manipulator arms 120, each manipulator arm 120 configured to couple to an instrument assembly 122. An instrument assembly 122 can include, for example, an instrument 126. As shown, each instrument assembly 122 is mounted to a distal portion of a respective manipulator arm 120. The distal portion of each manipulator arm 120 further includes a cannula mount 124 which is configured to have a cannula (not shown) mounted thereto. When a cannula is mounted to the cannula mount, a shaft of an instrument 126 passes through the cannula and into a worksite, such as a surgery site during a surgical procedure. The distal portion of each manipulator arm 120 further includes a cannula mount 124 which is configured to have a cannula (not shown) mounted thereto. When a cannula is mounted to the cannula mount, a shaft of an instrument 126 passes through the cannula and into a worksite, such as a surgery site during a surgical procedure. A force transmission mechanism 130 of the instrument assembly 122 can be connected to an actuation interface assembly 128 of the manipulator arm 120 that includes drive and/or other mechanisms controllable from workstation 102 to transmit forces to the force transmission mechanism 130 to actuate the instrument 126.

In various embodiments, one or more of instruments 126 can include an imaging device for capturing images (e.g., optical cameras, hyperspectral cameras, ultrasonic sensors, etc.). For example, one or more of instruments 126 can be an endoscope assembly that includes an imaging device, which can provide captured images of a portion of the worksite to be displayed via display unit 112.

In some embodiments, the manipulator arms 120 and/or instrument assemblies 122 can be controlled to move and articulate instruments 126 in response to manipulation of leader input devices 106 by operator 108, and in this way “follow” the leader input devices 106 through teleoperation. This enables the operator 108 to perform tasks at the worksite using the manipulator arms 120 and/or instrument assemblies 122. Manipulator arms 120 are examples of repositionable structures that a computer-assisted device (e.g., follower device 104) can include. In some embodiments, a repositionable structure of a computer-assisted device can include a plurality of links that are rigid members and joints that are movable components that can be actuated to cause relative motion between adjacent links. For a surgical example, the operator 108 can direct follower manipulator arms 120 to move instruments 126 to perform surgical procedures at internal surgical sites through minimally invasive apertures or natural orifices.

As shown, a control system 140 is provided external to workstation 102 and communicates with workstation 102. In other embodiments, control system 140 can be provided in workstation 102 or in follower device 104. As operator 108 moves leader input device(s) 106, sensed spatial information including sensed position and/or orientation information is provided to control system 140 based on the movement of leader input devices 106. Control system 140 can determine or provide control signals to follower device 104 to control the movement of manipulator arms 120, instrument assemblies 122, and/or instruments 126 based on the received information and operator input. In one embodiment, control system 140 supports one or more wired communication protocols, (e.g., Ethernet, USB, and/or the like) and/or one or more wireless communication protocols (e.g., Bluetooth, IrDA, HomeRF, IEEE 1102.11, DECT, Wireless Telemetry, and/or the like).

Control system 140 can be implemented on one or more computing systems. One or more computing systems can be used to control follower device 104. In addition, one or more computing systems can be used to control components of workstation 102, such as movement of a display unit 112.

As shown, control system 140 includes a processing system 150 and a memory 160 storing a control module 170. In some embodiments, control system 140 can include one or more processors, non-persistent storage (e.g., volatile memory, such as random access memory (RAM), cache memory), persistent storage (e.g., a hard disk, an optical drive such as a compact disk (CD) drive or digital versatile disk (DVD) drive, a flash memory, a floppy disk, a flexible disk, a magnetic tape, any other magnetic medium, any other optical medium, programmable read-only memory (PROM), an erasable programmable read-only memory (EPROM), a FLASH-EPROM, any other memory chip or cartridge, punch cards, paper tape, any other physical medium with patterns of holes, etc.), a communication interface (e.g., Bluetooth interface, infrared interface, network interface, optical interface, etc.), and numerous other elements and functionalities. The non-persistent storage and persistent storage are examples of non-transitory, tangible machine readable media that can include executable code that, when run by one or more processors (e.g., processing system 150), can cause the one or more processors to perform one or more of the techniques disclosed herein, including the processes of method 400, method 1100, and/or method 1200, described below. In addition, functionality of control module 170 can be implemented in any technically feasible software and/or hardware in some embodiments.

Each of the one or more processors of processing system 150 can be an integrated circuit for processing instructions. For example, the one or more processors can be one or more cores or micro-cores of a processor, a central processing unit (CPU), a microprocessor, a field-programmable gate array (FPGA), an application-specific integrated circuit (ASIC), a digital signal processor (DSP), a graphics processing unit (GPU), a tensor processing unit (TPU), and/or the like. Control system 140 can also include one or more input devices, such as a touchscreen, keyboard, mouse, microphone, touchpad, electronic pen, or any other type of input device.

A communication interface of control system 140 can include an integrated circuit for connecting the computing system to a network (not shown) (e.g., a local area network (LAN), a wide area network (WAN) such as the Internet, mobile network, or any other type of network) and/or to another device, such as another computing system.

Further, control system 140 can include one or more output devices, such as a display device (e.g., a liquid crystal display (LCD), a plasma display, touchscreen, organic LED display (OLED), projector, or other display device), a printer, a speaker, external storage, or any other output device. One or more of the output devices can be the same or different from the input device(s). Many different types of computing systems exist, and the aforementioned input and output device(s) can take other forms.

In some embodiments, control system 140 can be connected to or be a part of a network. The network can include multiple nodes. Control system 140 can be implemented on one node or on a group of nodes. By way of example, control system 140 can be implemented on a node of a distributed system that is connected to other nodes. By way of another example, control system 140 can be implemented on a distributed computing system having multiple nodes, where different functions and/or components of control system 140 can be located on a different node within the distributed computing system. Further, one or more elements of the aforementioned control system 140 can be located at a remote location and connected to the other elements over a network.

Some embodiments can include one or more components of a teleoperated medical system such as a da Vinci® Surgical System, commercialized by Intuitive Surgical, Inc. of Sunnyvale, California, U.S.A. Embodiments on da Vinci® Surgical Systems are merely examples and are not to be considered as limiting the scope of the features disclosed herein. For example, different types of teleoperated systems having follower devices at worksites, as well as non-teleoperated systems, can make use of features described herein.

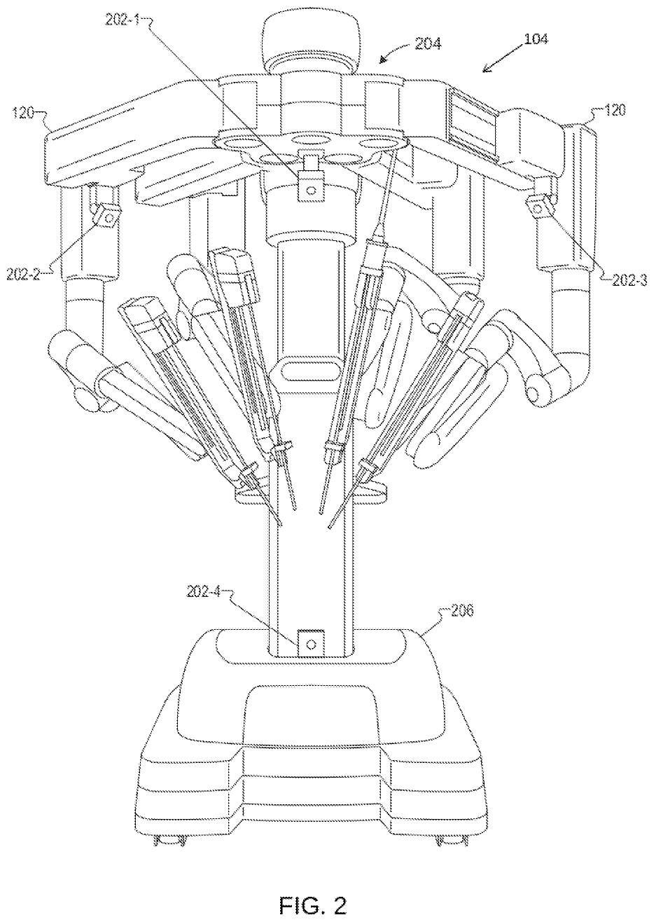

FIG. 2 is a simplified diagram of a follower device with a sensor system according to some embodiments. As shown, imaging devices 202 (imaging devices 202-1 through 202-4) are attached to portions of follower device 104. Although described herein with respect to imaging devices as a reference example, in some embodiments, a sensor system can include any technically feasible sensors, such as monoscopic and stereoscopic optical systems, ultrasonic systems, depth cameras such as cameras using time-of-flight sensors, LIDAR (light detection and ranging) sensors, etc. that are mounted on a computer-assisted system and/or elsewhere. For example, one or more sensors can be mounted on a base, on an orienting platform 204, and/or on one or more manipulator arms 120 of follower device 104. As another example, one or more sensors can be worn by an operator or mounted to a wall, a ceiling, the floor, or other equipment such as tables or carts.

Illustratively, imaging device 202-1 is attached to orienting platform 204 of follower device 104, imaging device 202-2 is attached to manipulating arm 120-1 of follower device 104, imaging device 202-3 is attached to manipulating arm 120-4 of follower device 104, and imaging device 202-4 is attached to a base 206 of follower device 104. In implementations in which follower device 104 is positioned proximate to a patient (e.g., as a patient side cart), placement of imaging devices 202 at strategic locations on follower device 104 provides advantageous imaging viewpoints proximate to a patient and areas around a worksite where a surgical procedure is to be performed on the patient.

The placements of imaging devices 202 on components of follower device 104 as shown in FIG. 2 are illustrative. Additional and/or alternative placements of any suitable number of imaging devices 202 and/or other sensors on follower device 104, other components of computer-assisted system 100, and/or other components (not shown) located in proximity to the follower device 104 can be used in sensor systems in other embodiments. Imaging devices 202 and/or other sensors can be attached to components of follower device 104, other components of computer-assisted system 100, and/or other components in proximity to follower device 104 in any suitable way. Additional computer-assisted systems including sensor systems that include sensors are described in International Application Publication No. WO 2021/097332, filed Nov. 13, 2020, and titled “Visibility Metrics in Multi-View Medical Activity Recognition Systems and Methods,” which is hereby incorporated by reference herein.

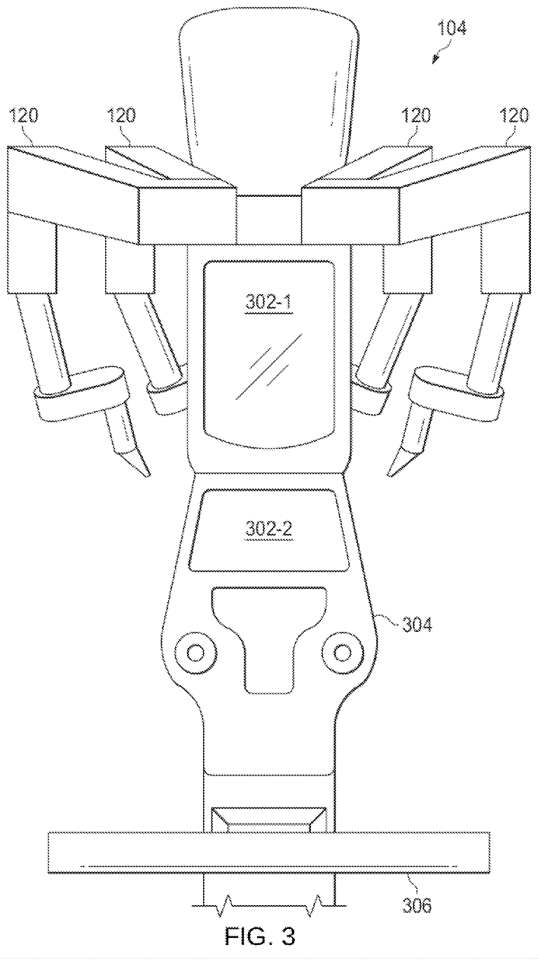

FIG. 3 is a simplified diagram of a follower device with a display system according to some embodiments. As shown, a user control interface (helm) 304 of follower device 104 includes display devices 302 (display devices 302-1 and 302-2). Illustratively, the user control interface 304 is attached to a repositionable structure of follower device 104 on a side opposite from manipulator arms 120. Display devices 302 are example output devices of follower device 104. In some embodiments, follower device 104 can include any technically feasible output device or devices. For example, one or more of display devices 302 can be cathode-ray tube (CRT) devices, liquid crystal display (LCD) devices, light-emitting diode (LED) devices, organic light-emitting diode (OLED) devices, quantum dot light-emitting diode (QLED) devices, plasma display devices, touchscreens, projectors, etc.

Illustratively, user control interface 304 also includes handlebars 306 that an operator can push or pull to reposition follower device 104 within an environment. In some embodiments, follower device 104 includes one or more actuators (e.g., one or more electric motors or servos) that drive the wheels (not shown) of follower device 104 based on input from the operator to assist the operator in repositioning follower device 104. For example, forces or torques applied by the operator on handlebars 306 can be used to determine a direction and speed of the one or more actuators. In some examples, user control interface 304 can include one or more buttons or other input devices (e.g., a joystick) to provide directional commands for controlling the one or more actuators. In some embodiments, repositioning of follower device 104 can be semi-autonomous or fully autonomous. In some other embodiments, follower device 104 does not include one or more actuators that assist the operator in repositioning follower device 104.

The placements of display devices 302 on follower device 104 as shown in FIG. 3 are illustrative. Additional and/or alternative placements of any suitable number of display devices 302 on follower device 104, other components of computer-assisted system 100, and/or other components (not shown) located in proximity to follower device 104 can be used in other embodiments. For example, one or more display devices can be attached to components of follower device 104, other components of computer-assisted system 100, and/or other components in proximity to follower device 104 in any suitable way. As further examples, one or more display devices can be included in a handheld device or a head-mounted device.

### Selecting a Deployment for a Computer-Assisted System

As discussed above, when a computer-assisted system having a follower device, such as the follower device 104 described in FIGS. 1-3, is used to perform a task at a worksite (e.g., an interior anatomy of a patient in a medical example), the follower device needs to be positioned within a sterile environment and oriented toward the patient so that the repositionable structures of the follower device can have suitable range of motion to control one or more instruments to access the worksite. In a typical arrangement, a non-surgeon operator controls the position and orientation of the computer-assisted system from a non-sterile side of the computer-assisted system that is separated from the sterile environment of the worksite and the patient by one or more sterile drapes. The non-surgeon operator is often assisted in this task by a user interface of the computer-assisted system that presents several options for selecting preselected deployments of the computer-assisted system, where each preselected deployment is associated with one or more target anatomical regions. Once the non-surgeon operator selects a desired deployment, the deployment is used to guide the non-surgeon operator in positioning and orienting the computer-assisted system relative to the patient. However, the responsibility for determining the target anatomy lies with the surgeon operator, who must communicate sufficient information about the target anatomy so that the non-surgeon operator can select the corresponding deployment. Importantly, selection of an incorrect deployment can lead to delays in a procedure or range of motion issues in the repositionable structures during the procedure.

In some instances, once a non-surgeon operator selects a desired deployment, the deployment is used to guide the non-surgeon operator in positioning and orienting the computer-assisted system relative to the patient. However, the responsibility for determining the target anatomy is the surgeon operator, who must communicate sufficient information about the target anatomy so that the non-surgeon operator can select the corresponding deployment. To do this effectively requires adequate communication between the surgeon operator on the sterile side of the computer-assisted system and the non-surgeon operator on the non-sterile side of the computer-assisted system. The effectiveness of this communication depends on the experience level of the non-surgeon operator in understanding the associations between the deployments and patient anatomy as well as the instructions from the surgeon as to the target anatomy for the procedure. This can lead to the selection of an incorrect deployment, which can result in delays in a procedure or range-of-motion issues in a repositionable structure during the procedure. To prevent the selection of incorrect deployments, automated selection of the appropriate deployment is helpful.

Consistent with some embodiments, an improved approach involves a computer-assisted system using sensor data to determine an imaging device orientation relative to a patient on an operating support and then using the imaging device orientation to automatically select a deployment that is used to guide a non-surgeon operator. In more detail, with a repositionable structure of the computer-assisted system positioned proximate a patient, an imaging device (e.g., an endoscope), which is not mounted to the computer-assisted system and is manually controlled by a surgeon operator, is inserted into the patient by the surgeon operator. The surgeon operator then positions and orients the imaging device so that the imaging device is capturing images of the target anatomy. Once the surgeon operator has positioned and oriented the imaging device toward the target anatomy, the computer-assisted system begins the deployment selection process. During the deployment selection process, the computer-assisted system determines a position of the repositionable structure relative to the patient and an orientation of the imaging device. The computer-assisted system uses the position of the repositionable structure and the orientation of the imaging device to determine a deployment for the repositionable structure of the computer-assisted system, such as a working direction. In some embodiments, the working direction for the repositionable structure is selected from a group of predetermined working directions. Further, in some embodiments, the working direction for the repositionable structure is selected based on a predetermined association between a particular working direction and a combination of the current position of the repositionable structure and the current orientation of the imaging device. This improved approach decreases the likelihood of the incorrect deployment of the computer-assisted system.

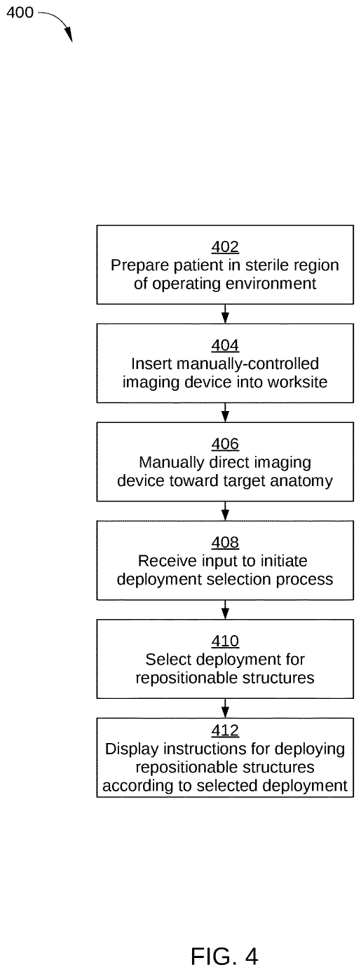

According to various embodiments, a deployment of a computer-assisted system is automatically selected to guide the non-surgeon operator to correctly position and orient a repositionable structure of the computer-assisted system. The deployment is selected based on a position of the repositionable structure relative to the patient and an imaging device orientation relative to a patient. Examples of such embodiments are described below in conjunction with FIGS. 4-12.

FIG. 4 is a simplified diagram of a method 400 for deploying a computer-assisted system according to some embodiments. According to some embodiments, method 400 can include one or more of the processes 402-412, which can be implemented, at least in part, in the form of executable code stored on a non-transitory, tangible, machine readable media that, when run on one or more processors (e.g., the processing system 150 in control system 140 of FIG. 1), can cause the one or more processors to perform one or more of the processes 402 412. In the embodiment described in conjunction with FIG. 4, method 400 is described with respect to a medical application and computer-assisted system 100. It will be apparent to one of ordinary skill in the art that method 4100 can be performed with any other suitable computer-assisted system. Further, in other embodiments, method 400 can be employed in other, non-surgical, applications, in which automatic selection of the deployment of any other suitable computer-assisted system can be beneficial.

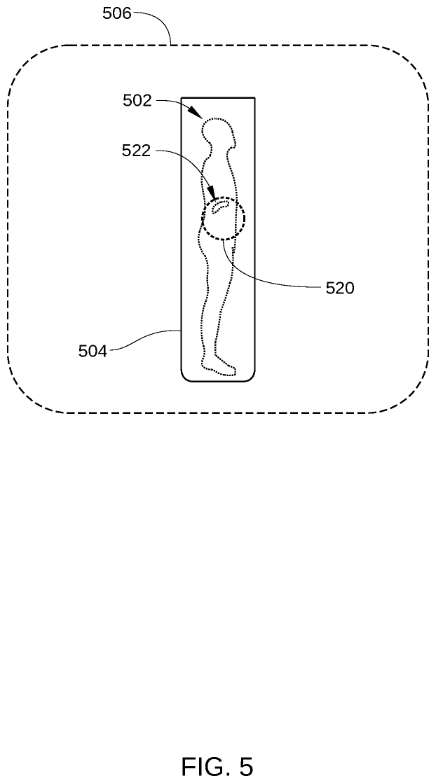

At process 402, a patient is prepared for a particular surgical procedure in a sterile region of an operating environment. For example, in some instances, the patient can be positioned on an operating table or other support that is located within a sterile region that is separated from a non-sterile region by one or more drapes or other barriers. In process 402, the patient can be positioned on the operating support in a specified orientation or bodily position, where the specified orientation or bodily position is associated with the particular surgical procedure. Examples of possible specified orientations or bodily positions include supine (face and abdomen facing upward), prone (face and abdomen facing downward), right lateral recumbent (right side oriented downward), and left lateral recumbent (left side oriented downward). An example embodiment of a patient positioned on an operating support is described below in conjunction with FIG. 5.

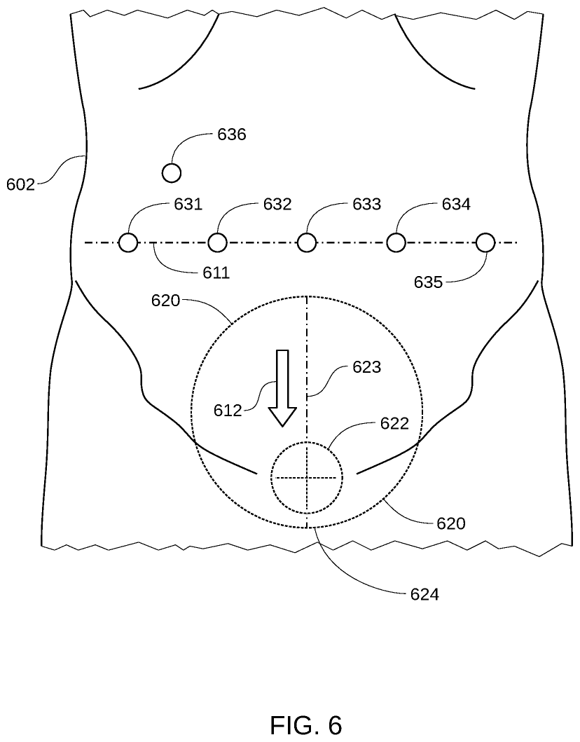

FIG. 5 is a simplified diagram of a patient 502 positioned on an operating support 504 according to some embodiments. As shown, patient 502 and operating support 504 are located within a sterile region 506 of an operating environment. Further, in the instance illustrated in FIG. 5, the bodily position of patient 502 is a left lateral recumbent position, in which the left side of patient 502 is oriented downward. Once the patient is positioned, a surgical worksite 520 is identified, for example by a surgeon operator (not shown) of computer-assisted system 100. In some embodiments, the surgical worksite 520 includes a target anatomy 522 and/or portions of the anatomy of patient 502 that require access by one or more instruments 126 (shown in FIG. 1). An example embodiment of a surgical workspace and target anatomy associated with a patient is described below in conjunction with FIG. 6.

FIG. 6 is a simplified diagram of a target anatomy 622 and an associated workspace 620 according to some embodiments. In the embodiment illustrated in FIG. 6, workspace 620 is a surgical workspace for a surgical procedure, and includes a target anatomy 622. In some embodiments, target anatomy 622 corresponds to a portion or region of the anatomy of a patient 602 where a midline 623 of workspace 620 intersects a far edge 624 of workspace 620. As shown, far edge 624 of workspace 620 is an edge of workspace 620 that is distal to ports 631-636, i.e., on an opposite side of workspace 620 than ports 631-636. In some embodiments, target anatomy 622 includes a pathology or other area or organ to be accessed in the surgical procedure.

Generally, workspace 620 includes a region of the anatomy of patient 602 that is to be accessed by one or more instruments 126 (shown in FIG. 1) of computer-assisted system 100, for example to complete the surgical procedure. For example, workspace 620 can correspond to or include a cavity that is created by insufflation of a gas into a region of the anatomy of patient 602 that surrounds target anatomy 622. During the surgical procedure, one or more instruments 126 can be positioned within such a cavity while the motions of instruments 126 are observed via one or more endoscopic cameras that are also positioned within workspace 620 and/or the cavity. Generally, the one or more instruments 126 and endoscopic cameras are inserted into the cavity via the one or more ports 631-636.

Each of ports 631-636 provide access to a cavity that is associated with workspace 620 and surrounds interior anatomy of patient 602. In some embodiments, each of ports 631 636 provides such access to a single instrument 126, such as an imaging device (e.g., an endoscope) or a minimally invasive surgical instrument (e.g., a surgical stapler, a suction irrigator, a gripper, and/or the like). In some embodiments, one or more of ports 631-636 provide access to two or more instruments 126. In the embodiment illustrated in FIG. 6, several of ports 631-636 are arranged along a line 611 that is perpendicular to an intended working direction 612 for computer-assisted system 100. In other embodiments, ports 631 636 can have a different arrangement than that depicted in FIG. 6. Generally, a port for receiving an instrument 126 is not placed between target anatomy 622 and another port for receiving an instrument 126. In some embodiments, working direction 612, also sometimes referred to as an “anatomy direction,” corresponds to a general direction in which the shafts of instrument 126 are oriented when accessing workspace 620 during the surgical procedure. In some embodiments, working direction 612 is parallel to midline 623 of workspace 620 and toward target anatomy 622.

Returning to FIG. 4, in process 402, as part of preparing the patient for the particular surgical procedure, one or more ports, such as ports 631-636 in FIG. 6, are appropriately placed for the particular procedure, for example by a surgeon-operator or other sterile operator making an incision and inserting a cannula or other guide tube for one or more instruments. As described above, the placement of ports proximate workspace 620 can be contingent on the location of the target anatomy associated with the particular surgical procedure within the workspace.

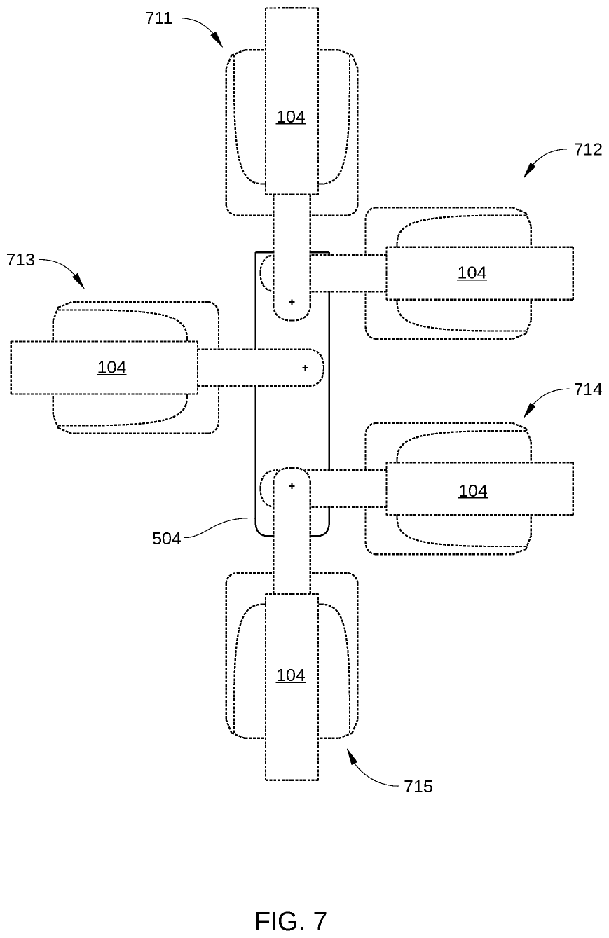

In some embodiments, as part of process 402, one or more repositionable structures of computer-assisted system 100, such as manipulator arms 120, are positioned relative to the patient. In some embodiments, the repositionable structures are positioned relative to the patient by the locating follower device 104 proximate the operating support on which the patient is positioned. Example embodiments of follower device 104 positioned proximate an operating support are described below in conjunction with FIG. 7.

FIG. 7 is a simplified diagram of various locations proximate operating support 504 at which follower device 104 can be positioned according to some embodiments. As shown, in some embodiments, follower device 104 can be positioned at a head location 711 relative to operating support 504. In some instances, positioning follower device 104 at head location 711 facilitates otorhinolaryngological (head and neck) surgical procedures. Additionally or alternatively, in some embodiments, follower device 104 can be positioned at a first side location 712 relative to operating support 504, which is near the head end of operating support 504. First side location 712 can be on the patient left or patient right side of operating support 504 (first side location 712 is shown on patient left side in FIG. 7). In some instances, positioning follower device 104 at first side location 712 facilitates thoracic and/or upper abdominal surgical procedures. Additionally or alternatively, in some embodiments, follower device 104 can be positioned at a second side location 713 relative to operating support 504, which is near a center region of operating support 504. Second side location 713 can be on the patient left or patient right side of operating support 504 (second side location 713 is shown on patient right side in FIG. 7). In some instances, positioning follower device 104 at second side location 713 facilitates renal, cardiac, and/or lower abdominal surgical procedures. Additionally or alternatively, in some embodiments, follower device 104 can be positioned at a third side location 714 relative to operating support 504, which is near the foot end of operating support 504. Third side location 714 can be on the patient left or patient right side of operating support 504 (third side location 714 is shown on patient left side in FIG. 7). In some instances, positioning follower device 104 at third side location 714 facilitates pelvic surgical procedures. Additionally or alternatively, in some embodiments, follower device 104 can be positioned at a foot location 715 relative to operating support 504, which is near the foot end of operating support 504. In some instances, positioning follower device 104 at foot location 715 facilitates pelvic surgical procedures.

Returning to FIG. 4, in some embodiments, as part of process 402, one or more repositionable structures of computer-assisted system 100 that are not mounted on a cart or other follower device 104 are positioned relative the patient. In such embodiments, the repositionable structures can be positioned in process 402 by movement along a track, such as a ceiling-mounted, wall-mounted, floor-mounted track, or patient support mounted track. Additionally or alternatively, in such embodiments, the repositionable structures can be positioned relative to the patient in process 402 by movement of the operating support relative to the repositionable structures. In either case, a surgeon operator can directly supervise or perform such positioning of the one or more repositionable structures relative to the patient.

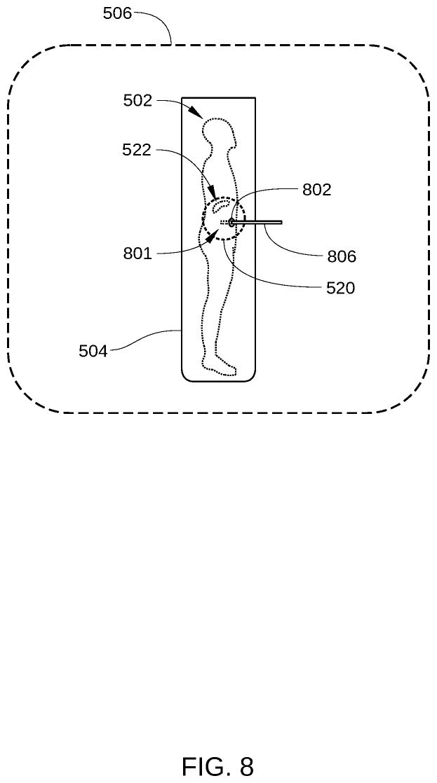

In process 404, a manually controlled imaging device is inserted into the worksite, for example by a surgeon operator. Because process 404 takes place prior to the final positioning of repositionable structures relative to the patient and the docking of instruments 126 to the patient, the insertion, orientation, and positioning of the imaging device is performed manually. An example embodiment of a manually controlled imaging device that is manually inserted into a worksite is described below in conjunction with FIG. 8.

FIG. 8 is a simplified diagram of a manually controlled imaging device 801 inserted into worksite 520 according to some embodiments. As shown, patient 502 is positioned on operating support 504, and manually controlled imaging device 801 is inserted into worksite 520, which includes target anatomy 522. For example, manually controlled imaging device 801 can be inserted into worksite 520 via a suitable port 802. In the embodiment illustrated in FIG. 8, manually controlled imaging device 801 is mounted on a shaft 806 that is compatible for use with port 802 or the cannula inserted into port 802. In some embodiments, imaging device 801 is kinematically decoupled from computer-assisted system 100, and therefore the position and orientation of imaging device 801 is not controlled by control system 140.

In some embodiments, manually controlled imaging device 801 is kinematically decoupled from the system (e.g., is not mounted to a robotic arm or repositionable structure of the computer-assisted system 100) during method 400, but can be kinematically coupled to a component of computer-assisted system 100 (e.g., via a robotic arm or other repositionable structure) before a surgical procedure commences. In some embodiments, the manually controlled imaging device 801 is also the same imaging device that is used and electronically registered with computer-assisted system 100 prior to starting method 400.

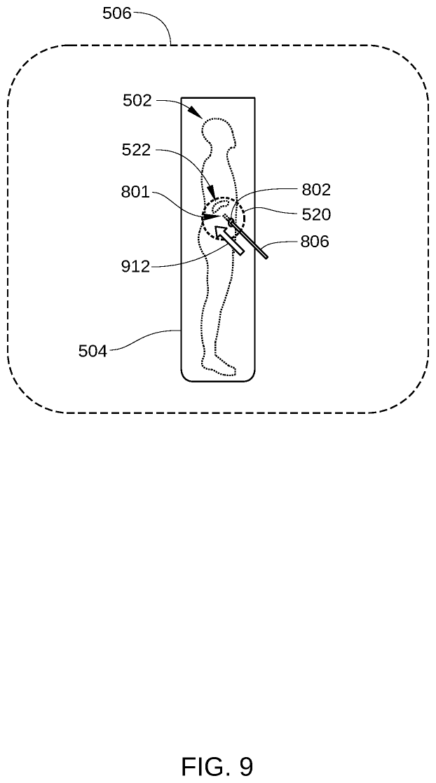

Returning to FIG. 4, in process 406, manually controlled imaging device 801 is directed manually toward target anatomy 522, as shown in FIG. 9. FIG. 9 is a simplified diagram of a manually controlled imaging device 801 being directed toward target anatomy 522 according to some embodiments. In some embodiments, a surgeon operator or other sterile operator disposed within sterile region 506 performs process 406, for example by monitoring images generated by manually controlled imaging device 801 and moving shaft 806 accordingly. In some embodiments, target anatomy 522 is detected automatically by computer-assisted system 100. For example, in some embodiments, computer-assisted system 100 automatically detects target anatomy 522 based on computer-vision analysis of image information received from manually controlled imaging device 801. In such embodiments, specific anatomical features and/or a pathology may be recognized via the computer-vision analysis and indicated to the surgeon operator or other sterile operator who is manually orienting manually controlled imaging device 801. In other embodiments, a surgeon operator orients manually controlled imaging device 801 toward target anatomy 522 based on image information received from manually controlled imaging device 801.

Manually controlled imaging device 801 can be any technically feasible imaging device suitable for use within worksite 520. In some embodiments, manually controlled imaging device 801 includes an endoscopic imaging device. In such embodiments, manually controlled imaging device 801 can be mounted on a straight shaft or an angled shaft. In some embodiments, manually controlled imaging device 801 includes a monoscopic or stereoscopic imaging device. Additionally or alternatively, in some embodiments, manually controlled imaging device 801 includes a light source, such as a visible, infrared, and/or ultraviolet light source. In some embodiments, manually controlled imaging device 801 includes an inertial measurement unit (IMU) mounted thereon that generates position and/or orientation information associated with manually controlled imaging device 801.

Returning to FIG. 4, in process 408, computer-assisted system 100 receives an input indicating that a deployment selection process is to be initiated. In some embodiments, the input is a user input associated with manually controlled imaging device 801, such as a button press or other input selection by the sterile operator controlling manually controlled imaging device 801. Additionally or alternatively, in some embodiments, the input can be a voice command by a sterile operator, a gesture by the sterile operator, and/or a command entered via a user interface. In such embodiments, the input selection can be performed via an input mechanism that is coupled to manually controlled imaging device 801 or included in follower device 104, such as leader input devices 106. Alternatively, in some embodiments, the input indicating that the deployment selection process is to be initiated is generated by computer-assisted system 100, for example in response to computer-assisted system 100 automatically detecting target anatomy 522 based on computer-vision analysis of worksite 520.

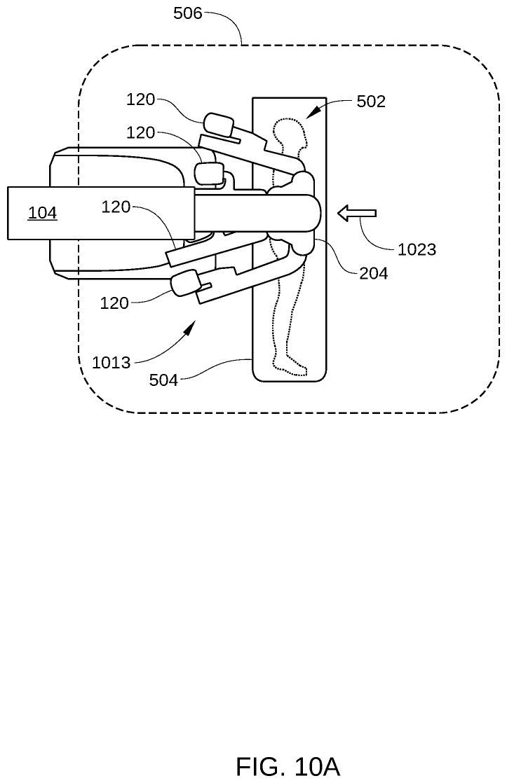

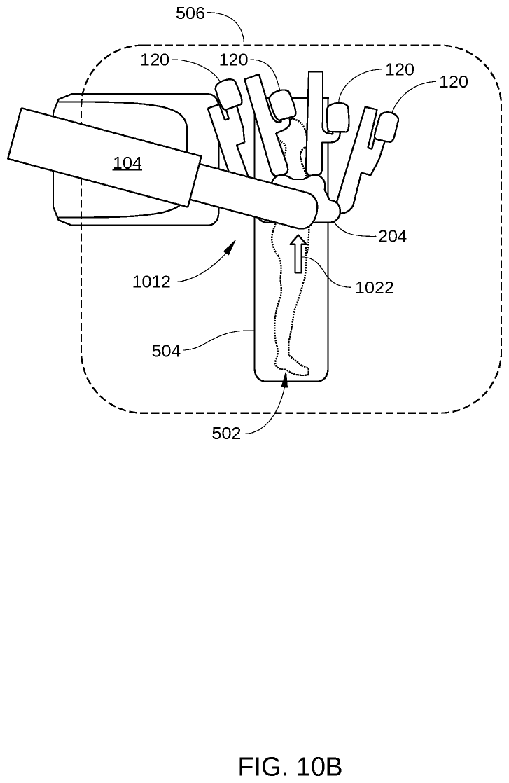

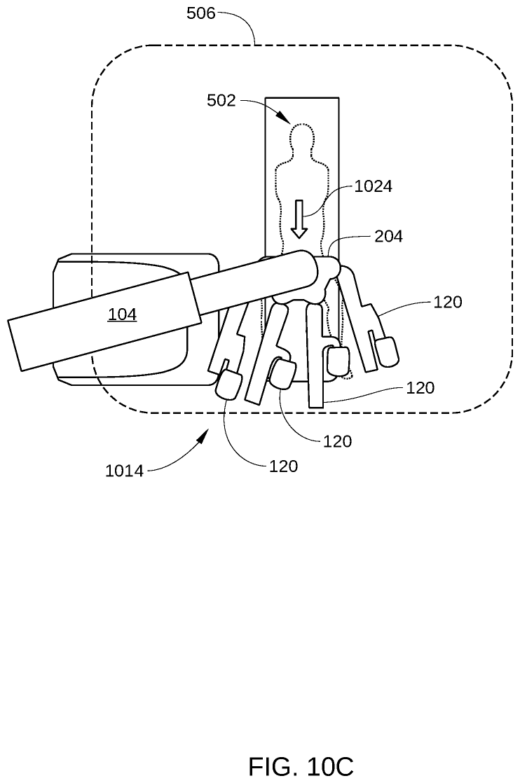

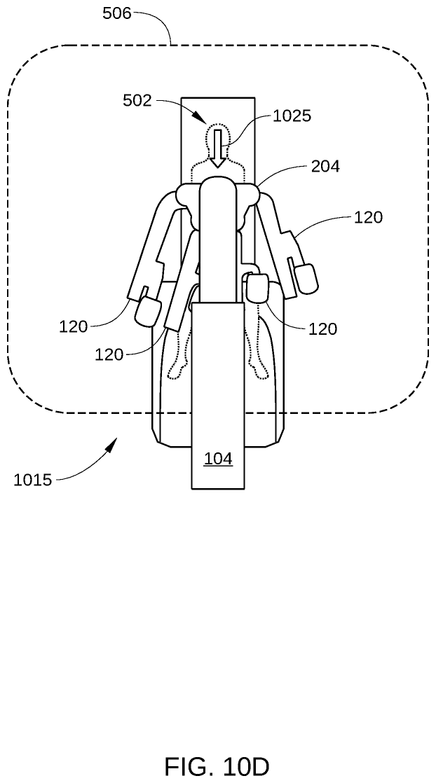

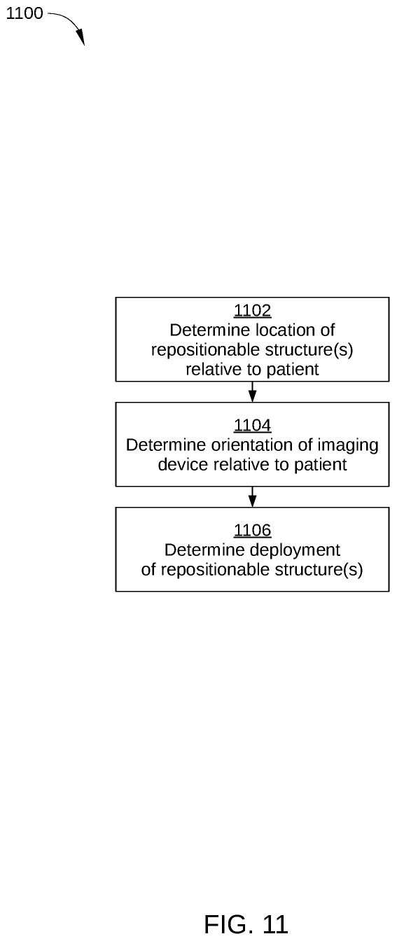

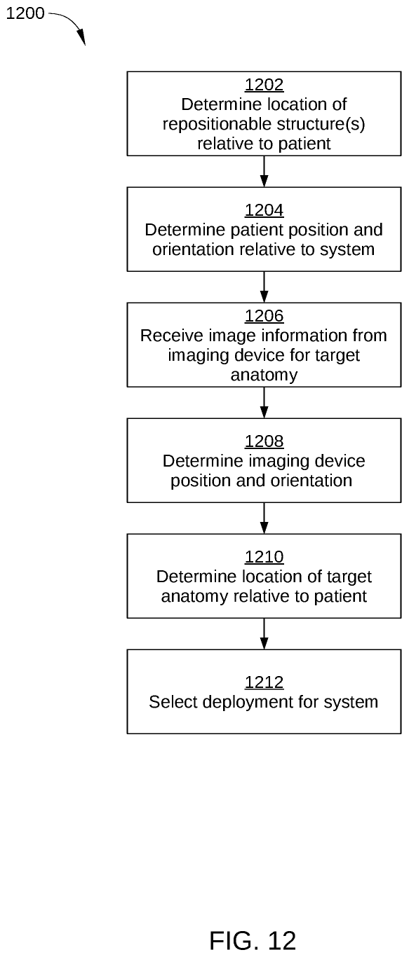

In process 410, computer-assisted system 100 selects a deployment of repositionable structures, such as manipulator arms 120, so that performance of the specified surgical procedure by the repositionable structures controlling the instruments will be well supported. In detail, computer-assisted system 100 selects a suitable working direction for computer-assisted system 100 based on the position of the repositionable structures relative to the patient and on an orientation of manually controlled imaging device 801 when computer-assisted system 100 receives the input indicating that a deployment selection process is to be initiated. It is noted that, when computer-assisted system 100 receives the input, manually controlled imaging device 801 is directed toward target anatomy 522 from port 802, as shown in FIG. 9. Therefore, manually controlled imaging device 801 is oriented in a direction that will be similar to a direction 912 in which the shafts of instrument 126 will be generally oriented (e.g., with an orientation that is within 45 to 60 degrees of direction 912) when accessing worksite 520 during the surgical procedure. Thus, when computer-assisted system 100 receives the input indicating that the deployment selection process is to be initiated, manually controlled imaging device 801 is oriented in a direction that is similar to the working direction for target anatomy 522 of patient 502. Example embodiments of various deployments of computer-assisted system 100 having different working directions are described below in conjunction with FIGS. 10A-10D. Example embodiments of computer-assisted system 100 selecting a deployment of repositionable structures are described below in conjunction with FIGS. 11 and 12.

In process 412, computer-assisted system 100 displays instructions for deploying repositionable structures according to selected deployment. In some embodiments, the selected deployment is displayed outside of sterile region 506, such as on display device 302-1 and/or 302-2 of follower device 104. Examples of displayed instructions include instructions directed to the repositioning of follower device 104 (e.g., move forward, rotate left, rotate right, move closer to patient head, move closer to patient feet) and instructions directed to positioning a portion of a repositionable structure of computer-assisted system 100 (e.g., rotate orienting platform 204 to face toward the patient feet, rotate orienting platform 204 to face toward patient head, rotate orienting platform 204 to face toward patient left, rotate orienting platform 204 clockwise or counterclockwise to a specified angle relative to the side of operating support 504, extend a boom supporting orienting platform 204 a specified distance, moving orienting platform 204 vertically, and the like.

Additionally or alternatively, in some embodiments, the selected deployment is displayed in the vicinity of computer-assisted system 100 and/or patient 502 via a wall-mounted, ceiling-mounted, or rack-mounted display device or another rack. Thus, a non-sterile operator can readily determine how to deploy the repositionable structures of computer-assisted system 100 for the surgical procedure without relying on verbal directions from a sterile operator. Alternatively, in some embodiments, in process 412, one or more of the base or the repositionable structure can be automatically moved to the selected deployment.

### Example Deployments for a Computer-Assisted System

FIGS. 10A-10D are simplified diagrams of example deployments that can be selected for the repositionable structures of computer-assisted system 100 according to some embodiments. In some embodiments, computer-assisted system 100 can select other deployments for the repositionable structures of computer-assisted system 100 in addition to the deployments illustrated in FIGS. 10A-10D.

FIG. 10A shows a deployment 1013 of manipulator arms 120 in which follower device 104 is positioned at a side location relative to operating support 504 and patient 502 that is consistent with second side location 713 of FIG. 7. In addition, in deployment 1013, orienting platform 204 is rotated so that manipulator arms 120 are positioned to operate with a lateral working direction 1023 as shown. According to various embodiments, when computer-assisted system 100 determines that follower device 104 is positioned at a side location consistent with second side location 713 and a manually controlled imaging device (not shown) is oriented in a direction substantially parallel to lateral working direction 1023, computer-assisted system 100 selects deployment 1013 for orienting platform 204.

FIG. 10B shows a deployment 1012 of manipulator arms 120 in which follower device 104 is positioned at a side location relative to operating support 504 and patient 502 that is consistent with first side location 712 of FIG. 7. In addition, in deployment 1012, orienting platform 204 is rotated so that manipulator arms 120 are positioned to operate with a “towards head” working direction 1022 as shown. According to various embodiments, when computer-assisted system 100 determines that follower device 104 is positioned at a side location consistent with first side location 712 and a manually controlled imaging device (not shown) is oriented in a towards head working direction 1022, computer-assisted system 100 selects deployment 1012 for orienting platform 204.

FIG. 10C shows a deployment 1014 of manipulator arms 120 in which follower device 104 is positioned at a side location relative to operating support 504 and patient 502 that is consistent with third side location 714 of FIG. 7. In addition, in deployment 1014, orienting platform 204 is rotated so that manipulator arms 120 are positioned to operate with a “towards feet” working direction 1024 as shown. According to various embodiments, when computer-assisted system 100 determines that follower device 104 is positioned at a side location consistent with third side location 714 and a manually controlled imaging device (not shown) is oriented in a towards feet working direction 1024, computer-assisted system 100 selects deployment 1014 for orienting platform 204.

FIG. 10D shows a deployment 1015 of manipulator arms 120 in which follower device 104 is positioned at a location relative to operating support 504 and patient 502 that is consistent with foot location 715 of FIG. 7. In addition, in deployment 1015, orienting platform 204 is rotated so that manipulator arms 120 are positioned to operate with a “towards feet” working direction 1025 as shown. According to various embodiments, when computer-assisted system 100 determines that follower device 104 is positioned at a location consistent with foot location 715 and a manually controlled imaging device (not shown) is oriented in a towards feet working direction 1025, computer-assisted system 100 selects deployment 1015 for orienting platform 204.

In the embodiments of manipulator arm deployments illustrated in FIG. 10 A-D, orienting platform 204 is rotated to one of several discrete positions so that manipulator arms 120 are positioned to operate with one of lateral working direction 1023, “towards head” working direction 1022, or “towards feet” working direction 1025. In other embodiments, in a deployment of manipulator arms 120, orienting platform 204 is aligned and oriented based on the orientation of a manually controlled imaging device, such as manually controlled imaging device 801 in FIG. 8. For example, in some embodiments, in a deployment of manipulator arms 120, orienting platform 204 is centered over the location of the manually controlled imaging device. Alternatively or additionally, in some embodiments, in a deployment of manipulator arms 120, orienting platform 204 is aligned with the orientation of the manually controlled imaging device, e.g., a front face vector of orienting platform 204 is aligned with the orientation of the manually controlled imaging device. In other embodiments, in a deployment of manipulator arms 120, follower device 104 is positioned relative to operating support based on the orientation and/or location of the manually controlled imaging device. For example, in some embodiments, in a deployment of manipulator arms 120, follower device 104 is horizontally centered at a horizontal location of the manually controlled imaging device.

### Selecting Deployment for a Computer-Assisted System According to a First Embodiment

FIG. 11 is a simplified diagram of a method 1100 for selecting a deployment for a computer-assisted system according to some embodiments. According to some embodiments, method 1100 can include one or more of the processes 1102-1106, which can be implemented, at least in part, in the form of executable code stored on a non-transitory, tangible, machine readable media that, when run on one or more processors (e.g., the processing system 150 in control system 140 of FIG. 1), can cause the one or more processors to perform one or more of the processes 1102-1106. In the embodiment described in conjunction with FIG. 11, method 1100 is described with respect to a medical application and computer-assisted system 100. It will be apparent to one of ordinary skill in the art that method 1100 can be performed with any other suitable robot-assisted system. Further, in other embodiments, method 1100 can be employed in other, non-surgical, applications, in which automatic selection of the deployment of any other suitable computer-assisted system can be beneficial.

At process 1102, computer-assisted system 100 determines the location of one or more repositionable structures relative to patient 502. In some embodiments, the repositionable structures include manipulator arms 120 and/or follower device 104. In some embodiments, computer-assisted system 100 uses imaging information received from one or more imaging, ranging, or tracking sensors associated with computer-assisted system 100 to determine the location of such repositionable structures. For example, in some embodiments, imaging information from imaging devices 202 is used in process 1102 to determine the location of repositionable structures of computer-assisted system 100. In some embodiments, computer-assisted system 100 performs computer-vision analysis to determine whether follower device 104 is disposed at one of head location 711, first side location 712, second side location 713, third side location 714, or foot location 715 relative to operating support 504. In some embodiments, point cloud techniques, object detection techniques, object segmentation techniques, and/or part segmentation techniques can be employed to identify objects or portions thereof. Such techniques can be machine learning-based or classical computer vision algorithms. Details for example computer vision techniques that utilize machine learning are described in PCT/US2021/059213, filed Nov. 12, 2021, and titled “VISIBILITY METRICS IN MULTI-VIEW MEDICAL ACTIVITY RECOGNITION SYSTEMS AND METHODS” which is hereby incorporated by reference herein.

In some embodiments, the one or more imaging, ranging, or tracking sensors include imaging devices 202. In some embodiments, the one or more imaging, ranging, or tracking sensors can be mounted on computer-assisted system 100, for example on a base, on a portion of a particular repositionable structure, or on orienting platform 204. Additionally or alternatively, the one or more imaging, ranging, or tracking sensors can be mounted elsewhere in the operating environment, for example worn by a surgeon operator or non-surgeon operator, or mounted to a surface external to computer-assisted system 100, such as a wall, ceiling, or floor, and/or mounted on tables, carts or other equipment.

At process 1104, computer-assisted system 100 determines the orientation of imaging device 801 relative to patient 502. In such embodiments, computer-assisted system 100 performs computer-vision analysis to determine the orientation of imaging device 801 relative to patient 502. In some embodiments, computer-assisted system 100 receives imaging information from similar imaging, ranging, or tracking sensors associated with computer-assisted system 100 that generate the imaging information received in process 1102. In some embodiments, computer-assisted system 100 uses one or more of the same object identification techniques employed in process 1102 to perform the computer-vision analysis of process 1104. Additionally or alternatively, in some embodiments, computer-assisted system 100 determines the orientation of manually controlled imaging device 801 based at least in part on position and/or orientation information received from an IMU mounted on manually controlled imaging device 801, such as pitch, yaw, and roll of shaft 806 on which manually controlled imaging device 801 is mounted. In some embodiments, computer-assisted system 100 selects the imaging device orientation from a group of discrete predetermined orientations, such as “towards patient left side,” “towards patient right side,” “towards patient head,” and “towards patient feet.”

At process 1106, computer-assisted system 100 determines a deployment of one or more repositionable structures of computer-assisted system 100, such as manipulator arms 120. In process 1106, computer-assisted system 100 determines the appropriate deployment based on the location of one or more repositionable structures determined in process 1102 and the orientation of manually controlled imaging device 801 determined in process 1104. Upon completion of process 1106, computer-assisted system 100 can perform process 412, in which computer-assisted system 100 displays instructions for deploying repositionable structures according to deployment selected in process 1106. In some embodiments, computer-assisted system 100 determines the deployment of the one or more repositionable structures based on the pose of manually controlled imaging device 801. In such embodiments, the pose can include both the orientation of manually controlled imaging device 801 and the position of manually controlled imaging device 801, for example, relative to computer assisted system 100.

In some embodiments, a table lookup is performed in process 1106 using the determined location of the repositionable structures as a first input and the current orientation of manually controlled imaging device 801 as a second input. Based on the first input and the second input, a predefined lookup table can then indicate the appropriate deployment. Thus, the lookup table indicates a predetermined association between a particular deployment and a particular combination of the current position of the repositionable structure relative to the patient and the current orientation of the imaging device relative to the patient.

In some embodiments, the number of possible locations of the repositionable structures is limited to a small number of discrete options, such as “on patient left side,” “on patient right side,” “at patient feet,” and “at patient head.” Similarly, in some embodiments, the number of possible orientations of manually controlled imaging device 801 is limited to a small number of discrete options, such as “towards patient left side,” “towards patient right side,” “towards patient head,” and “towards patient feet.” In such embodiments, the predefined lookup table can include a relatively small number of available output deployments. Examples of such output deployments include deployments in which orienting platform 204 is rotated so that manipulator arms 120 are positioned to operate with an appropriate working direction for the current location of follower device 104 relative to patient 502. In some embodiments, such working directions can include a direction that is parallel to the longitudinal axis of the patient (e.g., a head to feet axis) and toward a head of the patient, a direction that is parallel to the longitudinal axis of the patient and toward a foot of the patient, a direction that is perpendicular to the longitudinal axis of the patient and toward patient left, and/or a direction that is perpendicular to the longitudinal axis of the patient and toward patient right. In other embodiments, a larger number of possible discrete locations of the repositionable structures and/or possible discrete orientations of manually controlled imaging device 801 can be employed to determine an appropriate deployment using a table lookup. In some embodiments, such working directions can include a direction that is parallel to the imaging device orientation and/or a direction that is at an acute angle to a longitudinal axis of the patient.

### Selecting Deployment for a Computer-Assisted System According to a Second Embodiment

FIG. 12 is a simplified diagram of a method 1200 for selecting a deployment for a computer-assisted system according to some embodiments. According to some embodiments, method 1200 can include one or more of the processes 1202-1212, which can be implemented, at least in part, in the form of executable code stored on a non-transitory, tangible, machine readable media that, when run on one or more processors (e.g., the processing system 150 in control system 140 of FIG. 1), can cause the one or more processors to perform one or more of the processes 1202-1212. In the embodiment described in conjunction with FIG. 12, method 1200 is described with respect to a medical application and computer-assisted system 100. It will be apparent to one of ordinary skill in the art that method 1200 can be performed with any other suitable robot-assisted system. Further, in other embodiments, method 1200 can be employed in other, non-surgical, applications, in which automatic selection of the deployment of any other suitable computer-assisted system can be beneficial.

At process 1202, computer-assisted system 100 determines the location of one or more repositionable structures relative to patient 502. In some embodiments, process 1202 is consistent with process 1102 in FIG. 11.

At process 1204, computer-assisted system 100 determines the position and orientation of patient 502 relative to computer-assisted system 100 in a common frame of reference. In such embodiments, computer-assisted system 100 can map the location of the one or more repositionable structures and the position and orientation of patient 502 to the common frame of reference, such as a world or system coordinate frame or a patient-centric coordinate frame. In some embodiments, computer-assisted system 100 uses imaging information received from one or more of the same imaging, ranging, or tracking sensors associated with computer-assisted system 100 that are used to determine the location of repositionable structures in process 1202.

At process 1206, computer-assisted system 100 receives image information from manually controlled imaging device 801. Generally, the image information received in process 1206 can include imaging of target anatomy 522.

At process 1208, computer-assisted system 100 determines the position and orientation of manually controlled imaging device 801. In such embodiments, computer-assisted system 100 can then map the position and orientation of manually controlled imaging device 801 to a common frame of reference, such as a world or system coordinate frame or a patient-centric coordinate frame. Thus, computer-assisted system 100 can map the position and orientation of manually controlled imaging device 801 to a common frame of reference along with the location of the one or more repositionable structures and the position and orientation of patient 502.

In some embodiments, computer-assisted system 100 determines the position and orientation of manually controlled imaging device 801 based on the image information received in process 1206 of target anatomy 522. In such embodiments, computer-assisted system 100 uses one or more of the same object identification techniques employed in process 1102 to perform computer-vision analysis of the imaging of target anatomy 522. Additionally or alternatively, in some embodiments, computer-assisted system 100 determines the orientation of manually controlled imaging device 801 based at least in part on position and/or orientation information received from an IMU mounted on manually controlled imaging device 801, such as pitch, yaw, and roll of shaft 806 on which manually controlled imaging device 801 is mounted. Additionally or alternatively, in some embodiments, computer-assisted system 100 determines the orientation and position of manually controlled imaging device 801 based at least in part on a location where manually controlled imaging device 801 is inserted into the anatomy of patient, such as the location of port 802. Additionally or alternatively, in some embodiments, computer-assisted system 100 determines the orientation and position of manually controlled imaging device 801 based at least in part on an insertion depth of shaft 806, as indicated by the same imaging, ranging, or tracking sensors associated with computer-assisted system 100 that are used to determine the location of repositionable structures in process 1202. Additionally or alternatively, in some embodiments, computer-assisted system 100 determines the orientation and position of manually controlled imaging device 801 based at least in part on an insertion angle of shaft 806, as indicated by the same imaging, ranging, or tracking sensors associated with computer-assisted system 100.

At process 1210, computer-assisted system 100 determines the location of target anatomy 522 relative to the position and orientation of patient 502. In such embodiments, computer-assisted system 100 determines a particular sector or region of the anatomy of patient 502 in which target anatomy 522 is disposed. For example, in some embodiments, the anatomy of patient 502 can be divided into predefined sectors or quadrants, and in process 1210 computer-assisted system 100 determines in which predefined sector or quadrant target anatomy 522 is disposed. Thus, in such embodiments, computer-assisted system 100 determines in which of a discrete number of available locations of target anatomy 522 target anatomy 502 is located. In some embodiments, computer-assisted system 100 determines in which predefined sector or quadrant target anatomy 522 is disposed based on various inputs, including the position and orientation of patient 502 determined in process 1204, the orientation of manually controlled imaging device 801 determined in process 1208, and/or the image information received in process 1206 of target anatomy 522.

At process 1212, computer-assisted system 100 determines a deployment of one or more repositionable structures of computer-assisted system 100, such as manipulator arms 120. In process 1212, computer-assisted system 100 determines the appropriate deployment based on specific inputs, such as the location of one or more repositionable structures determined in process 1202, the position and orientation of patient 502 determined in process 1204, the location of target anatomy 522 determined in process 1210, and/or the orientation of manually controlled imaging device 801 determined in process 1208. In some embodiments, computer-assisted system 100 selects the appropriate deployment of the one or more repositionable structures in process 1212 from a discrete number of available deployments. Thus, in such embodiments, each combination of specific inputs has a predetermined association with a discrete deployment of one or more repositionable structures of computer-assisted system 100. Examples of such deployments include deployments in which orienting platform 204 is rotated so that manipulator arms 120 are positioned to operate with an appropriate working direction for the current location of follower device 104 relative to patient 502. Upon completion of process 1212, computer-assisted system 100 can perform process 412, in which computer-assisted system 100 displays instructions for deploying repositionable structures according to deployment selected in process 1212.

In the embodiment described above, computer-assisted system 100 uses sensor data to determine an imaging device orientation relative to a patient on an operating support while the imaging device is directed toward and capturing images of a target anatomy. Computer-assisted system 100 then uses the imaging device orientation to automatically select a deployment of a repositionable structure that is used to guide a non-surgeon operator. In other embodiments, computer-assisted system 100 employs a multi-target approach, in which multiple target anatomies are identified and stored, and a repositionable structure can be changed from a first deployment to a second deployment based on one or more of the stored target anatomies. In some embodiments, each of the multiple target anatomies can be disposed in a different sector or quadrant of patient anatomy. In some embodiments, the repositionable structure is changed from the first deployment to the second deployment during a procedure, such as during a change in a phase of a procedure from a first target anatomy to a second target anatomy, in response to an operator instruction, and/or in response to a change in a mode of computer-assisted system 100. Alternatively or additionally, in some embodiments, a single deployment is selected based on the multiple target anatomies, so that computer-assisted system 100 selects a deployment suitable for procedures associated with two or more of the target anatomies.

Although illustrative embodiments have been shown and described, a wide range of modification, change and substitution is contemplated in the foregoing disclosure and in some instances, some features of the embodiments may be employed without a corresponding use of other features. One of ordinary skill in the art would recognize many variations, alternatives, and modifications. Thus, the scope of the invention should be limited only by the following claims, and it is appropriate that the claims be construed broadly and, in a manner, consistent with the scope of the embodiments disclosed herein.

Any and all combinations of any of the claim elements recited in any of the claims and/or any elements described in this application, in any fashion, fall within the contemplated scope of the present invention and protection.

The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments.

Aspects of the present embodiments may be embodied as a system, method or computer program product. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “module,” a “system,” or a “computer.” In addition, any hardware and/or software technique, process, function, component, engine, module, or system described in the present disclosure may be implemented as a circuit or set of circuits. Furthermore, aspects of the present disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Aspects of the present disclosure are described above with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine. The instructions, when executed via the processor of the computer or other programmable data processing apparatus, enable the implementation of the functions/acts specified in the flowchart and/or block diagram block or blocks. Such processors may be, without limitation, general purpose processors, special-purpose processors, application-specific processors, or field-programmable gate arrays.

The flowchart and block diagrams in the figures illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

While the preceding is directed to embodiments of the present disclosure, other and further embodiments of the disclosure may be devised without departing from the basic scope thereof, and the scope thereof is determined by the claims that follow.

1. A computer-assisted system comprising:
a repositionable structure; and
a processing system;
wherein the processing system is configured to perform the steps of:
determining a location of the repositionable structure relative to a patient on an operating support;
determining an imaging device orientation of an imaging device relative to the patient, the imaging device configured to be mountable to the repositionable structure;
determining a deployment for the repositionable structure based on the imaging device orientation and the location of the repositionable structure relative to the patient; and
causing one or more instructions for configuring the repositionable structure to be displayed.

2. The computer-assisted system of claim 1, wherein the imaging device is kinematically decoupled from the computer-assisted system.

3. (canceled)

4. The computer-assisted system of claim 1, wherein determining the imaging device orientation is performed in response to the processing system determining that the imaging device is directed toward a target anatomy of the patient.

5-7. (canceled)

8. The computer-assisted system of claim 1, wherein determining the deployment for the repositionable structure comprises determining a working direction for the repositionable structure based on the imaging device orientation.

9. The computer-assisted system of claim 8, wherein determining the working direction based on the imaging device orientation comprises selecting the working direction from a group of predetermined working directions, wherein the group of predetermined working directions includes a direction toward a left side of the operating support, a direction toward a right side of the operating support, a direction toward a foot of the patient, and a direction toward a head of the patient.

10-11. (canceled)

12. The computer-assisted system of claim 8, wherein determining the working direction comprises one of a direction that is parallel to the imaging device orientation, a direction that is at an acute angle to a longitudinal axis of the patient, a direction that is parallel to the longitudinal axis of the patient and toward a head of the patient, a direction that is parallel to the longitudinal axis of the patient and toward a foot of the patient, a direction that is perpendicular to the longitudinal axis of the patient and toward patient left, or a direction that is perpendicular to the longitudinal axis of the patient and toward patient right.

13. The computer-assisted system of claim 1, wherein:
the repositionable structure is mounted on a follower device of the computer-assisted system, and
determining the location of the repositionable structure relative to the patient comprises selecting a location of the follower device from a group of locations consisting of a location proximate a left side of the operating support, a location proximate a right side of the operating support, a location proximate a foot of the patient, and a location proximate a head of the patient.

14. (canceled)

15. The computer-assisted system of claim 1, wherein determining the deployment for the repositionable structure comprises determining the deployment based on a predetermined association between the location of a target anatomy of the patient and the deployment.

16. The computer-assisted system of claim 15, wherein the predetermined association between the location of the target anatomy and the deployment is included in a plurality of predetermined associations.

17. The computer-assisted system of claim 16, wherein each predetermined association is between a different location of the target anatomy and an available deployment for the repositionable structure.

18. (canceled)

19. The computer-assisted system of claim 1, further comprising determining the location of a target anatomy of the patent based on a location at which the imaging device is inserted into an interior anatomy of the patient.

20. The computer-assisted system of claim 19, wherein determining the location of the target anatomy is further based on:
an insertion depth into the interior anatomy of the patient of an instrument that includes the imaging device; or
an insertion angle into the interior anatomy of the patient of an instrument that includes the imaging device.

21-23. (canceled)

24. The computer-assisted system of claim 1, wherein the one or more instructions for configuring the repositionable structure comprise at least one of instructions directed to positioning of a follower device comprising the repositionable structure or instructions directed to positioning a portion of the repositionable structure of the computer-assisted system.

25-27. (canceled)

28. A method comprising:
determining, by a processing system, a location of a repositionable structure of a computer-assisted system relative to a patient on an operating support;
determining, by the processing system, an imaging device orientation of an imaging device relative to the patient, the imaging device configured to be mountable to the repositionable structure;
determining, by the processing system, a deployment for the repositionable structure based on the imaging device orientation and the location of the repositionable structure relative to the patient; and
causing, by the processing system, one or more instructions for configuring the repositionable structure to be displayed.

29. The method of claim 28, wherein the imaging device is kinematically decoupled from the computer-assisted system.

30. (canceled)

31. The method of claim 28, wherein determining the imaging device orientation is performed in response to the processing system determining that the imaging device is directed toward a target anatomy of the patient.

32-34. (canceled)

35. The method of claim 28, wherein determining the deployment for the repositionable structure comprises determining a working direction for the repositionable structure based on the imaging device orientation.

36. The method of claim 35, wherein determining the working direction based on the imaging device orientation comprises selecting the working direction from a group of predetermined working directions, wherein the group of predetermined working directions includes a direction toward a left side of the operating support, a direction toward a right side of the operating support, a direction toward a foot of the patient, and a direction toward a head of the patient.

37-38. (canceled)

39. The method of claim 35, wherein determining the working direction comprises one of a direction that is parallel to the imaging device orientation, a direction that is at an acute angle to a longitudinal axis of the patient, a direction that is parallel to the longitudinal axis of the patient and toward a head of the patient, a direction that is parallel to the longitudinal axis of the patient and toward a foot of the patient, a direction that is perpendicular to the longitudinal axis of the patient and toward patient left, or a direction that is perpendicular to the longitudinal axis of the patient and toward patient right.

40-54. (canceled)

55. A non-transitory machine-readable medium comprising a plurality of machine-readable instructions which when executed by one or more processors associated with a computer-assisted device, are adapted to cause the computer-assisted device to perform a method comprising:
determining, by a processing system, a location of a repositionable structure of a computer-assisted system relative to a patient on an operating support;
determining, by the processing system, an imaging device orientation of an imaging device relative to the patient, the imaging device configured to be mountable to the repositionable structure;
determining, by the processing system, a deployment for the repositionable structure based on the imaging device orientation and the location of the repositionable structure relative to the patient; and
causing, by the processing system, one or more instructions for configuring the repositionable structure to be displayed.
